(19) United States
(12) Patent Application Publication
(10) Pub. No.: US 2012/0265213 A1
(43) Pub. Date: Oct. 18, 2012

(54) SURGICAL DISTRACTION INSTRUMENT FOR LAMINOPLASTY

(75) Inventors: Jens Beger, Tuttlingen (DE); Ralph Linke, Steisslingen (DE); Petr Suchomel, Liberec (CZ)

(73) Assignee: Aesculap AG, Tuttlingen (DE)

(21) Appl. No.: 13/439,253

(22) Filed: Apr. 4, 2012

(30) Foreign Application Priority Data
Apr. 12, 2011 (DE) 10 2011 001 997

(51) Int. Cl. A61B 17/66; A61B 17/88

(52) U.S. Cl. 606/102; 606/105

(57) ABSTRACT

A surgical instrument and instrumentation are suggested, with which expansion of the vertebral canal of vertebrae is possible with less stress for the patient than with operating procedures customary up to now, this instrument having proximally a gripping part and distally two branches which can be moved relative to one another with tool elements held thereon as well as a lever mechanism arranged between the gripping part and the branches, wherein the tool elements are held with the lever mechanism so as to be movable relative to one another from a closed rest position into an open spread position (distraction position).

The invention relates to a surgical distraction instrument for use in laminoplasty, with which the vertebral arch of a vertebra is cut through, thereby forming an incision gap, and the incision gap is enlarged with the distraction instrument for the purpose of inserting an implant, as well as surgical instrumentation comprising such a distraction instrument.

The vertebral canals of the vertebrae of the spinal column form the so-called canalis vertebralis or spinal canal, in which the spinal cord, enclosed by the spinal meninx, is accommodated.

The spinal cord as part of the central nervous system may be prevented from functioning properly if pressure is exerted on the spinal cord, for example, with the occurrence of spinal canal stenosis. This may have several causes, for example the existence of spondylosis or ossification of the posterior longitudinal ligament.

This may be remedied by increasing the size of the vertebral canal of the vertebra or vertebrae of the spine that is or are affected so that the spinal cord has more space available and can therefore evade the pressure.

An overview of the therapeutic options commonly used to date can be found, for example, in F. Meyer et al., Deutsches Ärtzeblatt, year 105, issue 20, pages 366 to 372. In addition to the ventral methods, various dorsal methods, namely laminectomy with and without fusion and laminoplasty, are used. Ventral methods may, where applicable, also be used in combination with dorsal methods.

Of the various dorsal methods, laminoplasty works with the least surgical interventions in the bone substance.

To date, various laminoplasty operating techniques have been proposed. The two most important of these are described in the literature as so-called single-door or double-door techniques. An overview of these and an assessment of the effects to be expected with regard to pressure relief and expansion of the spinal canal is included, for example, in the publication by Wang, Xiang-Yang et al. in SPINE, Vol. 31, No. 24, 2006, pages 2863 to 2870.

In the so-called single-door technique, also called open-door technique, the lamina is cut through on one side of the vertebra with an incision gap, whereas on the other side of the lamina a groove is made without cutting the vertebral arch.

The area of the vertebral arch with the groove acts like a hinge during the subsequent opening of the vertebral canal and allows the vertebral arch to be opened, which involves fracture of the bone substance. The vertebral arch remains joined to the vertebral body by the periosteum and the collagenous fibers of the bone substance.

In the so-called double-door technique, the spinous process of a vertebra is cut through or completely removed and a groove is made in the lamina on both sides of the spinous process, with the areas of the vertebral arch containing the grooves again acting as hinges. The vertebral canal is now opened by swinging apart the two vertebral arch sections with the associated spinous process parts, if still existing, and the bone substance in the area of the hinges likewise fractures. Here, too, the vertebral arch sections remain joined to the vertebral body by the periosteum and the collagenous fibers of the bone substance.

With both techniques, the vertebral canal of the vertebrae is fixed in the opened state by implants. In addition to the body's own bone chip, a hydroxyapatite spacer or the like is used as implant material.

In spite of the reduced surgical intervention in the bone substance in comparison with other dorsal methods, a significantly increased rate of subsequent neck pain is still regarded as a disadvantage of laminoplasty, as is restricted mobility of the cervical spine, which is often observed.

The object of the invention is to suggest a surgical instrument or instrumentation, with which expansion of the spinal canal of vertebrae is possible with less stress for the patient.

This object is accomplished by a surgical instrument with the features of claim 1 and instrumentation according to claim 14.

The instrument according to the invention is preferably used in methods, with which, in contrast to the single-door and double-door techniques of laminoplasty discussed above, only a single separating incision will be made at the vertebral arch, in particular in the area of the spinous process, on the one hand, and thus a detachment of muscle from the spinal column will be avoided to a great extent or even essentially altogether. The enlargement of the vertebral arch will be achieved by means of elastic/plastic deformation thereof and so any fracture of the bone substance of the lamina can be avoided.

Owing to the inherent viscoelastic properties of the bone substance, the elastic/plastic deformation occurs additionally with the method in accordance with the invention but without this leading to any fracture of the bone substance. It is preferable for the enlargement not to be forced abruptly, but to be performed gradually, so that the viscoelastic properties of the bone substance can come into play. This can happen continuously or in small steps of, for example, approximately 0.5 mm to approximately 3 mm each. Typically, a gap enlargement of approximately 10 to approximately 12 mm within approximately 10 sec to approximately 5 min, in particular approximately 30 sec to approximately 3 min, can be achieved in this way.

This elastic/plastic deformation for enlargement of the incision gap with minimized risk of fracture of the bone substance is achieved, in particular, by limiting the forces used for the enlarging to about 500 N or less, in particular about 300 N or less.

This counteracts the problem of the laminoplasty techniques used to date, which first necessitate extensive surgical interventions on the muscles extending parallel to the spine, which also mean considerable stress for the patient in the postoperative phase.

The incision gap may be produced with very different tools. For example, the incision gap may be made with an ultrasonic osteotome, with incision gaps of about 1 mm or less resulting.

Other techniques use high-speed drills or burrs (craniotome), with which somewhat wider incision gaps are obtained, for example ranging from approximately 2 mm to approximately 3 mm.

Another technique uses the so-called T-saw or Gigli saw, but here there is a certain difficulty in initially introducing the saw without injuring the spinal meninx. The same applies to use of the craniotome.

Surprisingly, in spite of the minimal surgical interventions, it is possible with the laminoplasty technique described to obtain access to the spinal canal and expansion thereof, which are comparable to the conventional single-door and double-door techniques as regards the clinical results to be expected.

The distraction instruments according to the invention, in particular in the form of distraction forceps, are used for the elastic/plastic enlargement of the incision gap and these preferably have tool elements which have a contact surface at their distal end for the positioning of the instrument in the gap.

The contact surfaces are designed, in particular, at the distal or rather free end of the tool elements as laterally projecting flanges or angled points. These preferred distraction instruments according to the invention are introduced into the incision gap in a sagittal direction, wherein the contact surfaces of the tool elements come to rest on the spinal canal side of the vertebral arch sections and thus form a guide for the instrument.

The contact surfaces further ensure a secure seating of the distraction instrument according to the invention during the enlargement of the incision gap.

During the introduction of the distraction instrument, the incision gap can be widened slightly with a thin blade or a raspatory.

The forces required for enlarging the incision gap are typically approximately 70 N to approximately 200 N, in particular approximately 80 N to approximately 150 N, for enlargement of the gap by approximately 5 mm to approximately 12 mm, determined, in each case, at the end of the incision gap on the spinal canal side.

In the majority of cases, the elastic/plastic enlargement is achieved without fracturing the bone material. If a fracture has been observed during enlargement by up to approximately 10 mm, it related to the fracture in the area of the spinous process.

The distraction forces are, therefore, preferably introduced as close as possible to or better in the area of the vertebral arch and not in the area of the spinous process.

At the distal end, the tool elements preferably have a stop element which serves for the positioning of the implant in the incision gap. The stop element is preferably designed as a flange which projects forwards and, in particularly preferred embodiments, adjoins directly to the flanges which project to the side and form the contact surfaces.

The stop element allows a secure placement of the implant in the incision gap; in particular, it can preclude too deep an insertion of the implant, during which the ventral end area of the implant would jut into the spinal canal.

A signal, which is haptically clearly discernible when the ventral end area of the implant impinges on the stop element and indicates that the desired insert depth for the implant has been reached, results for the surgeon during insertion of the implant, preferably along the branches of the distraction instrument according to the invention.

As a result, a secure placement of the implant is ensured even when the view of the incision gap is obstructed, for example, by fluid in the wound.

Distraction instruments according to the invention which are even more preferred have tool elements, the cross section of which is of a wedge-shaped design in the end area to be inserted into the incision gap. As a result, the centering of the instrument during its introduction into the incision gap in a sagittal direction is facilitated. Also, with this embodiment of the distraction instrument according to the invention, the tool may be inserted into an incision gap, the original gap width of which is very small, without needing to resort to additional aids.

This purpose is served by an additional feature of a distraction instrument according to the invention, with which the tool elements are designed so as to taper conically towards their end in the direction of the closing direction.

In order to achieve a defined enlargement of the incision gap, the tool elements have, in the opening direction, a bending strength which corresponds to a distraction force of approximately 300 N or more without any plastic deformation thereby occurring at the tool elements.

A better view of the incision gap is provided by the preferred measure of arranging the longitudinal axis of the branches holding the tool elements at an obtuse angle in relation to the longitudinal axis or plane of pivoting of the gripping part.

The obtuse angle is preferably selected in the range of approximately 110° to approximately 160°, for example approximately 135°.

The branches holding the tools preferably have on their upper side, i.e. in the direction, in which the stop elements, which are optionally present, project, guide elements which make a guided insertion of the implant into the incision gap possible.

The instrument according to the invention preferably has a locking device which is mounted, in particular, on the gripping element and by means of which the tool elements can be fixed relative to one another in one or more predetermined relative positions in the opening direction.

As a result, an important aid is provided for the step-wise enlargement of the incision gap for the optimum utilization of the viscoelastic properties of the bone substance of the vertebral arch.

Alternatively or in combination with this further development of the distraction instrument according to the invention, this can be provided with a device for determining the distance of the end areas of the tool elements relative to one another. As a result, the gap width of the incision gap, which has been achieved distally, can be easily read, for example, in the region of the gripping part.

In addition or alternatively to the two measures discussed above, it may be provided for the distraction instrument according to the invention to comprise a force measuring device so that the enlargement of the incision gap can be carried out whilst avoiding forces which are too great. The force measuring device preferably has a display for a predetermined maximum force which is, where possible, not to be exceeded, for example of 300 N or less.

The present invention relates, in addition, to surgical instrumentation for laminoplasty which includes as central element a distraction instrument of the present invention as described above. In addition, the surgical instrumentation according to the invention includes a holding instrument for an implant to be inserted which makes it easier for the surgeon to insert this into its correct position in the incision gap.

One preferred holding instrument has a gripping part which is arranged proximally, a shaft adjoining thereto and a holding device arranged at the distal end of the shaft for releasably fixing an implant in position.

The holding device preferably has a slide which is designed complementarily to the guide elements of the distraction element or which has guide surfaces which cooperate with the branches holding the tool elements of the distraction element and thus allow a guided insertion of the implant into the incision gap.

Prior to the insertion of an implant, a spacer can, where required, be inserted temporarily into the enlarged incision gap and this is likewise a component of a preferred instrumentation according to the invention.

The spacer preferably has a spacer element which is preferably of a U-shaped design and maintains access to the spinal canal. In this case, as well, the spacer is preferably introduced into the incision gap to such a depth that it comes to rest in the region of the vertebral arch and not or not exclusively in the region of the spinous process. The orientation of the U shape of the spacer element relative to the vertebra is, in this respect, cranial/caudal.

The spacer is often already held in the incision gap adequately by the elastically/plastically spread vertebral arches. If any unintentional movement of the spacer is to be avoided, this can be secured to the skin of the patient or via an optional shaft to a retractor which keeps the surgical wound open and is, optionally, likewise part of the instrumentation according to the invention.

The inserted spacer with U-shaped spacer element allows a substantially unobstructed view of and access to the spinal cord and the spinal meninx.

The enlargement of the vertebral canal of a vertebra with the instrumentation according to the invention also creates access to cranially or caudally adjacent vertebral canals to such an extent that a decompression of the spinal cord can be achieved there with the so-called undercutting technique.

It is, for example, thus possible by means of the instrumentation according to the invention to achieve an enlargement of the vertebral canals of the C4 and C6 vertebrae and a decompression in the entire section of the cervical spine from C3 to C7.

The two incision surfaces defining the incision gap in the vertebral body are preferably enlarged to a spacing of approximately 5 mm to approximately 15 mm, measured at the end of the incision gap on the spinal canal side.

The incision surfaces of the incision gap are fixed in the enlarged position by instruments or implants in a way similar to that also possible in connection with the prior art operating method.

The material from which the implant is made is preferably a biocompatible plastic material, in particular PEEK or titanium or a titanium alloy. The body's own bone chips are also suitable.

Implants made of plastic, in particular PEEK, are preferably provided with an osteointegrative coating on the surfaces contacting the bone material. This coating is preferably applied as a microporous pure titanium coating using the VPS process (Plasmapore technique) or as a hydroxyapatite coating.

The plastic implants are preferred over titanium implants as they are compatible with the MRT procedure. This is of particular importance for the postoperative phase. MRT compatibility also exists with the osteointegratively coated plastic implants described above.

The implant will preferably have a wedge shape so that as full a surface contact as possible of the surfaces of the wedge-shaped body on the incision surfaces of the enlarged incision gap can be achieved, the incision surfaces initially being arranged parallel once the incision gap has been formed but being inclined relative to one another in the shape of a wedge following the elastic enlargement of the incision gap.

These and further advantages of the invention will be explained in greater detail in the following on the basis of the drawings. These show in detail:

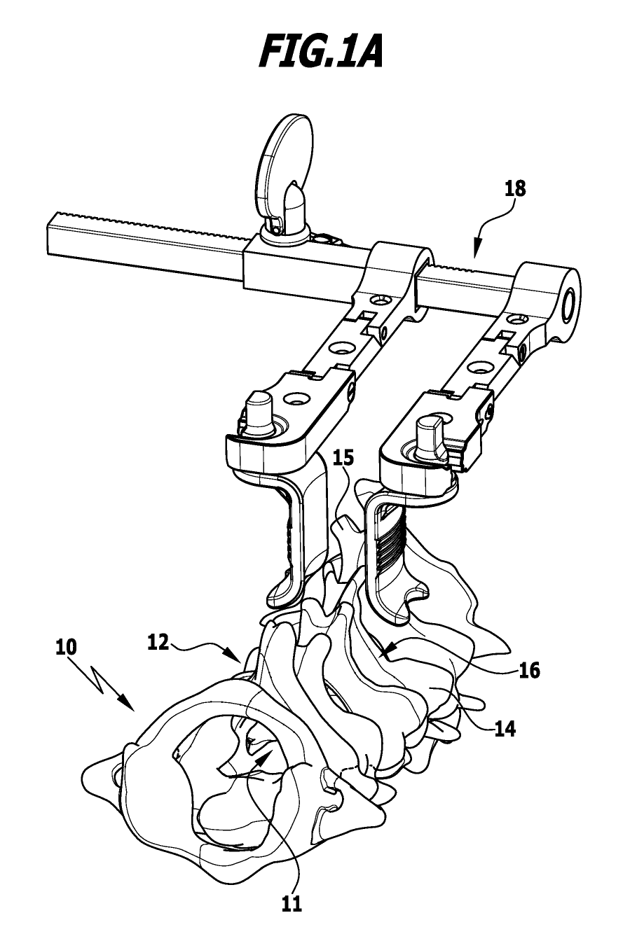

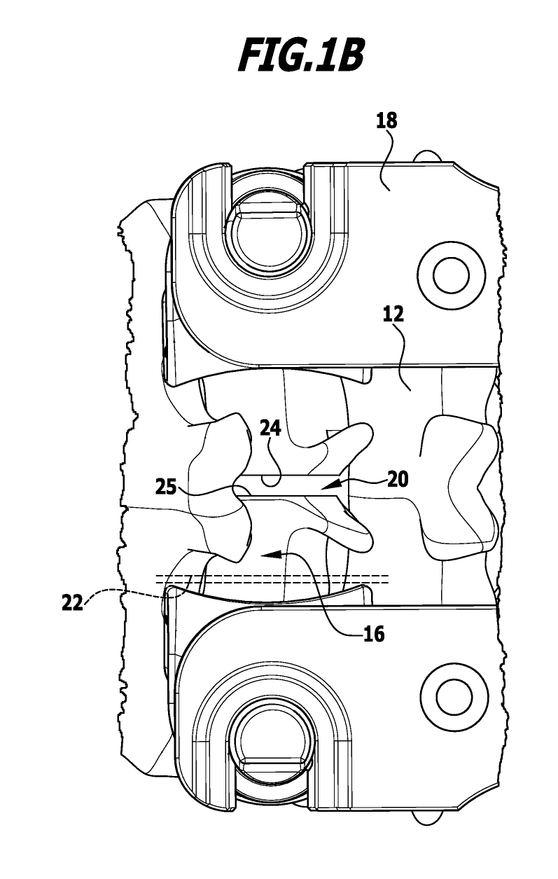

FIGS. 1A and 1B part of the cervical spine with a cervical vertebra provided with an incision gap in accordance with the invention in a perspective and plan view;

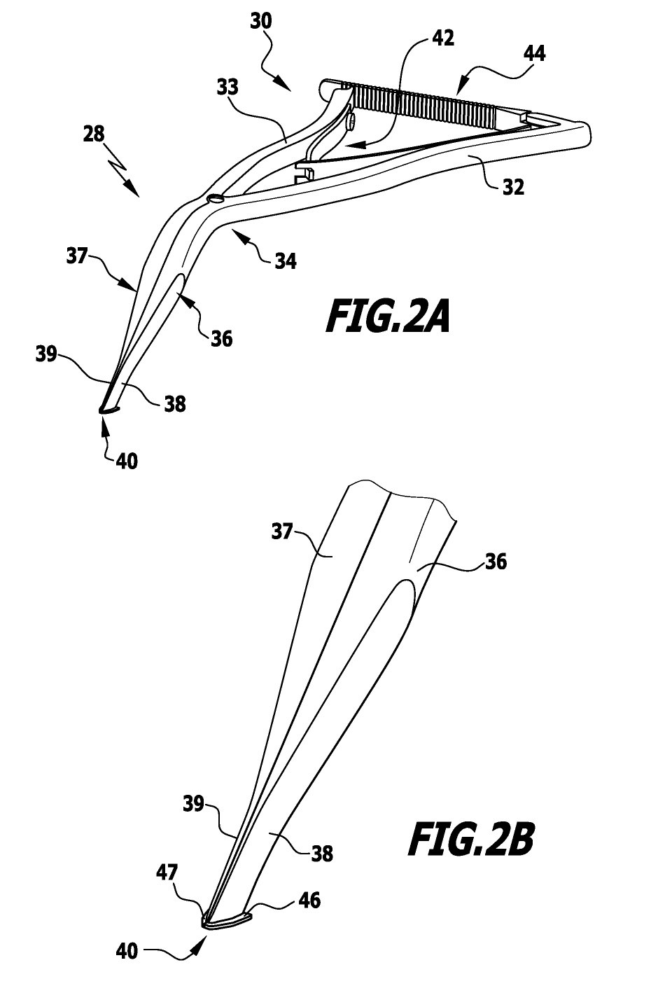

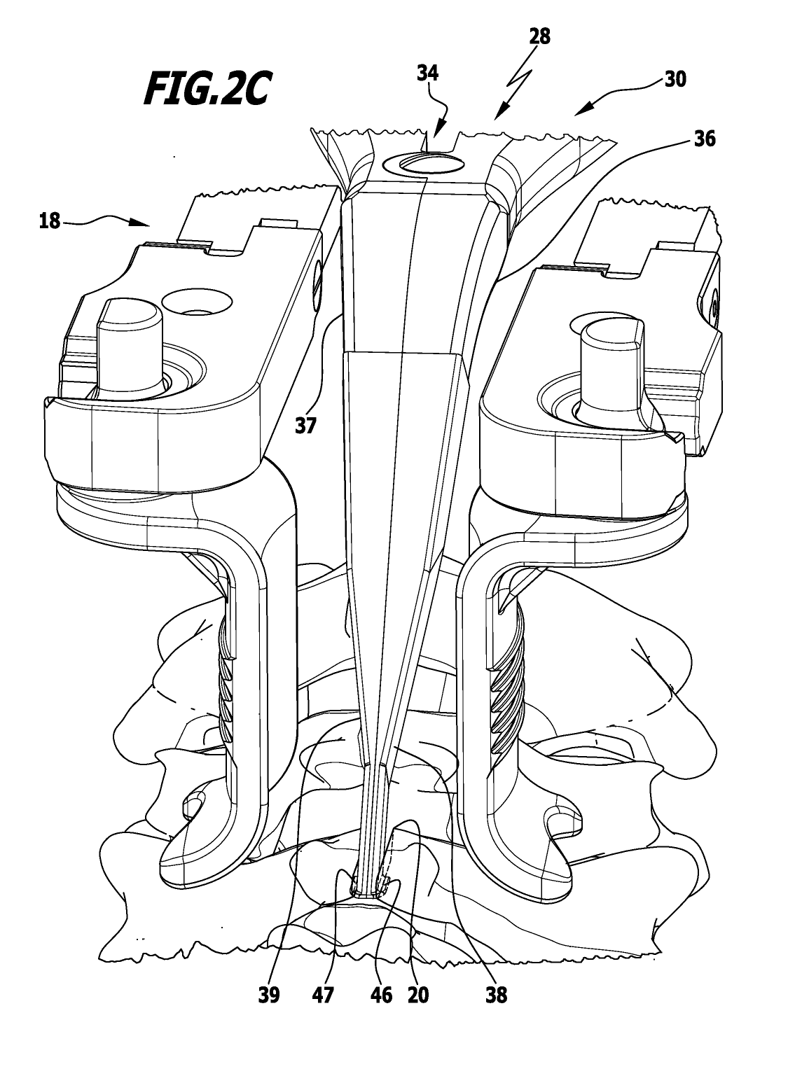

FIG. 2A a first embodiment of a distraction instrument according to the invention;

FIG. 2B a detail of the distraction instrument according to the invention of FIG. 2A;

FIG. 2C the illustration of the cervical vertebra of FIG. 1B during the enlargement with an inserted distraction instrument according to the invention;

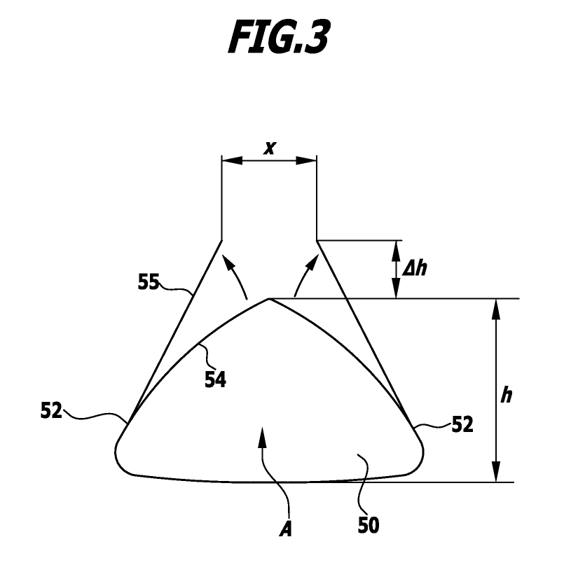

FIG. 3 a schematic sectional view through a vertebral canal elastically enlarged in accordance with the invention;

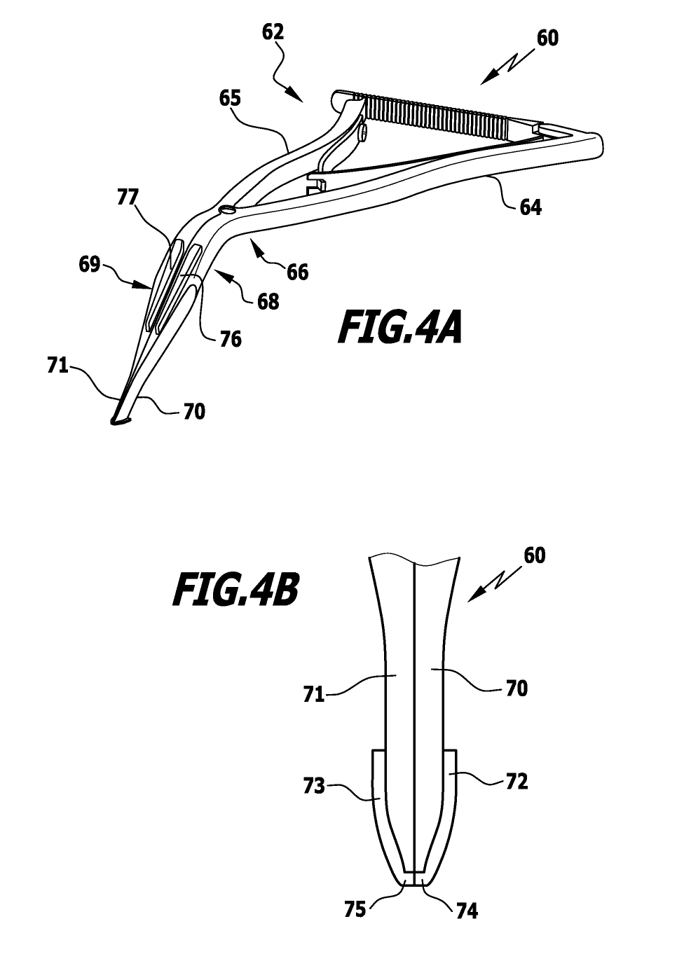

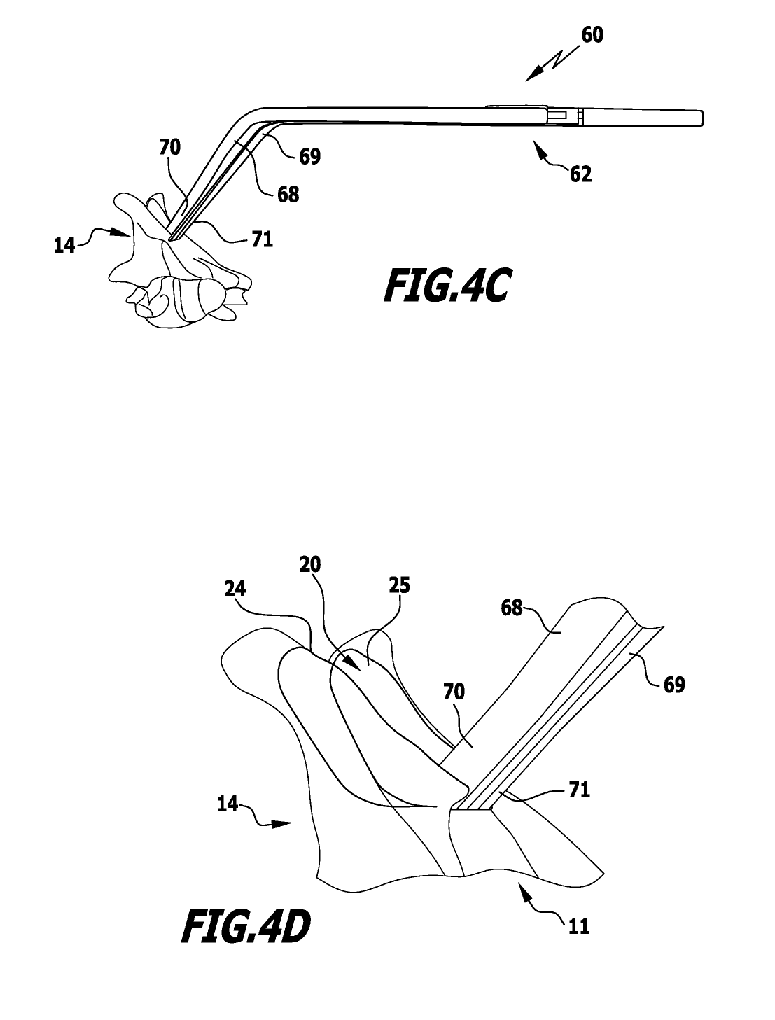

FIGS. 4A to 4D overall illustration and detailed views of an additional embodiment of a distraction instrument according to the invention;

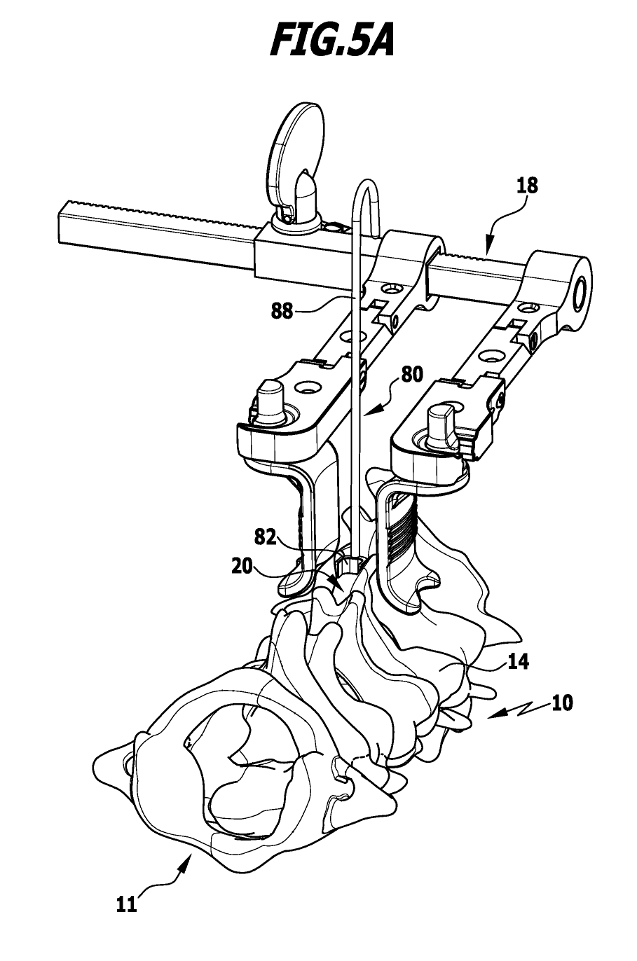

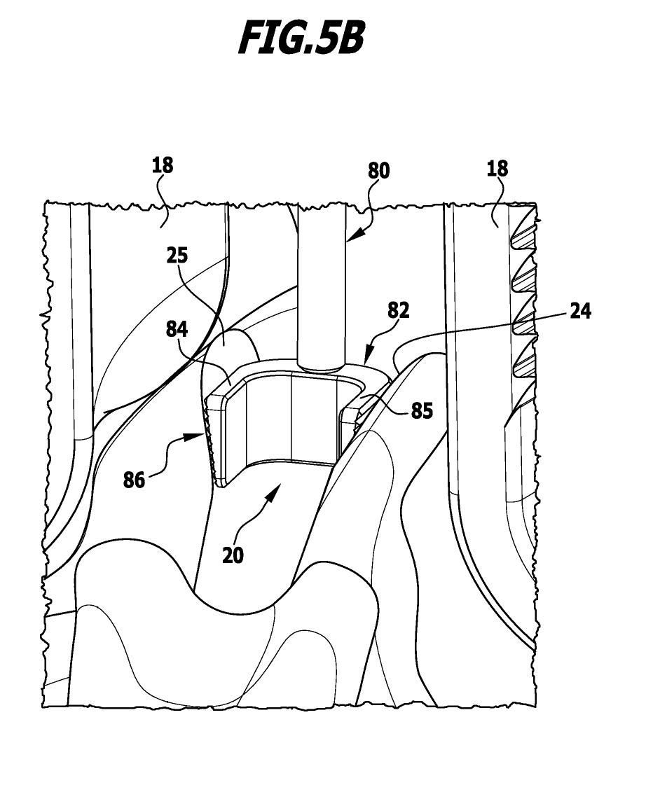

FIGS. 5A and 5B vertebra with enlarged incision gap and an inserted spacer according to the invention;

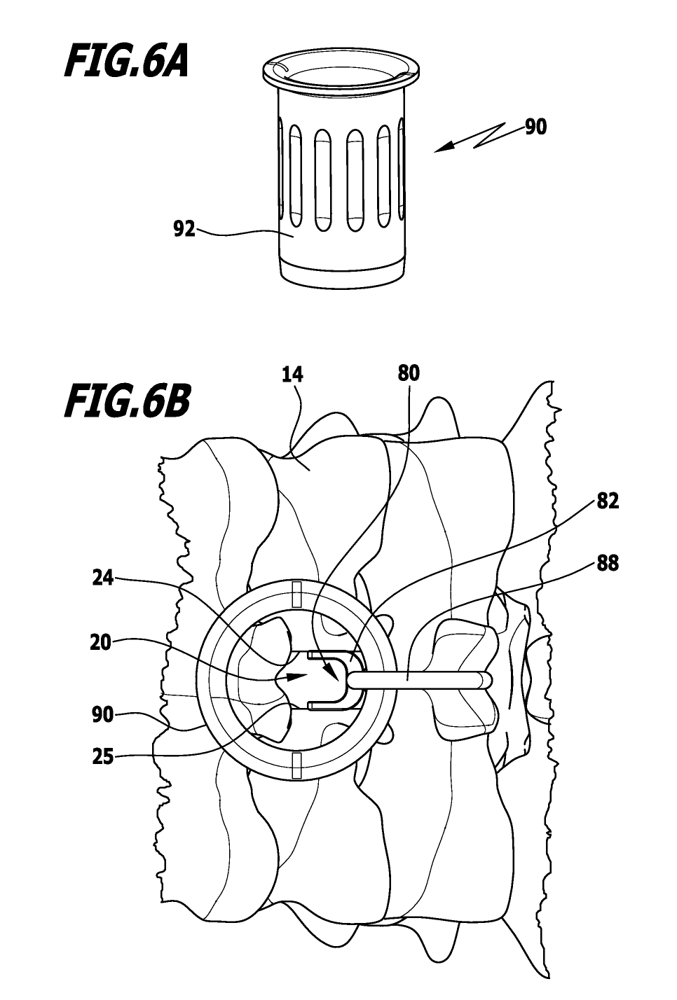

FIGS. 6A and 6B a perspective illustration of an alternative retractor as well as a plan view of an enlarged incision gap of a vertebra in accordance with FIGS. 5A and 5B with the alternative retractor in a plan view;

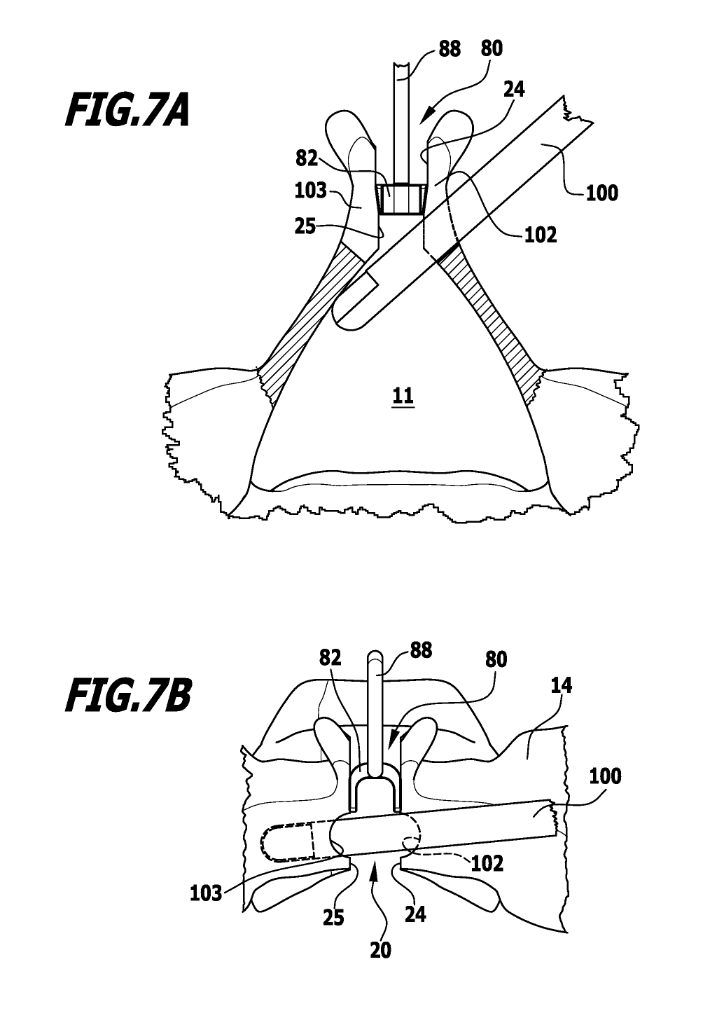

FIGS. 7A and 7B an operative treatment in the vertebral canal with a spacer according to the invention inserted in the incision gap;

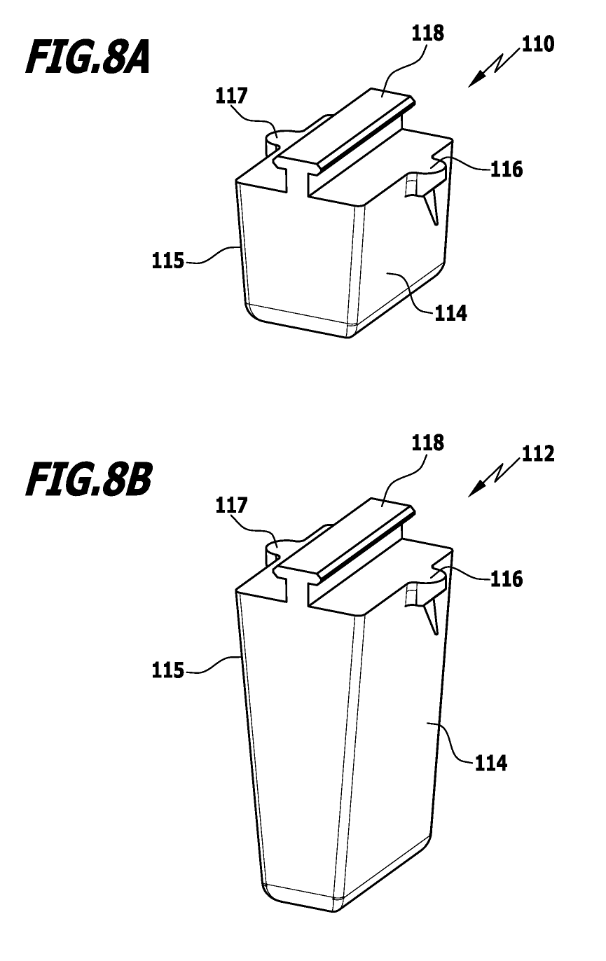

FIGS. 8A and 8B basic shapes of a first embodiment of an implant in two variations;

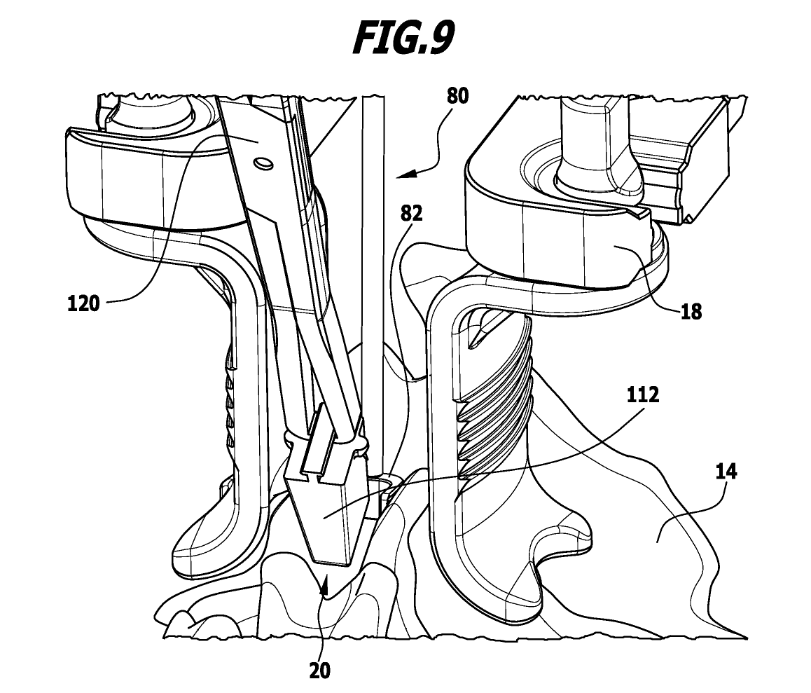

FIG. 9 the insertion of an implant into the incision gap held open by a spacer according to the invention;

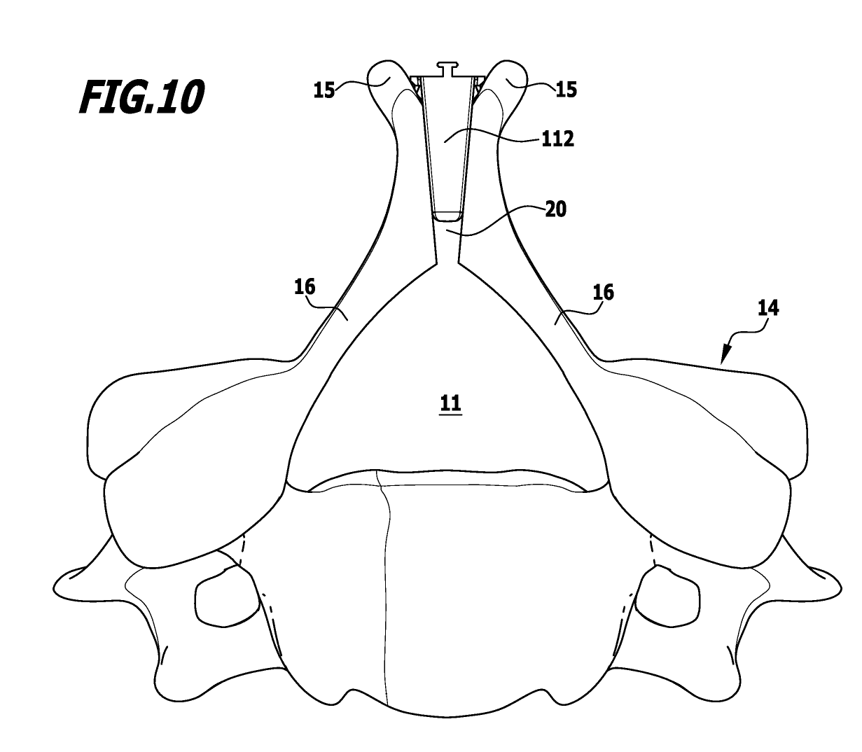

FIG. 10 the implant of FIG. 8B inserted in the incision gap of the vertebra;

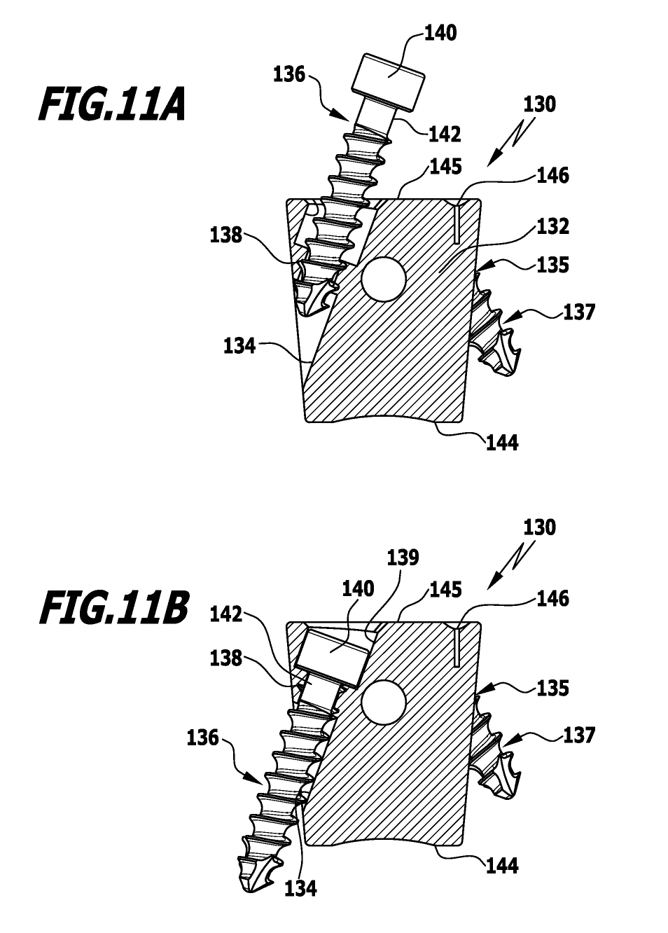

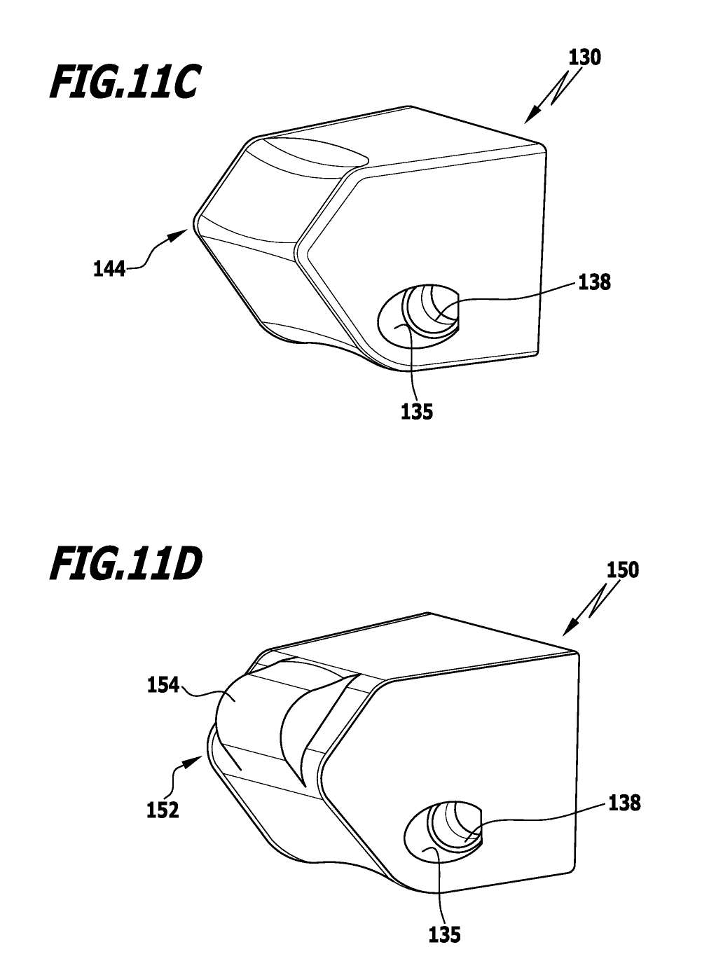

FIGS. 11A to 11D further variations of the implant in a perspective and sectional illustration;

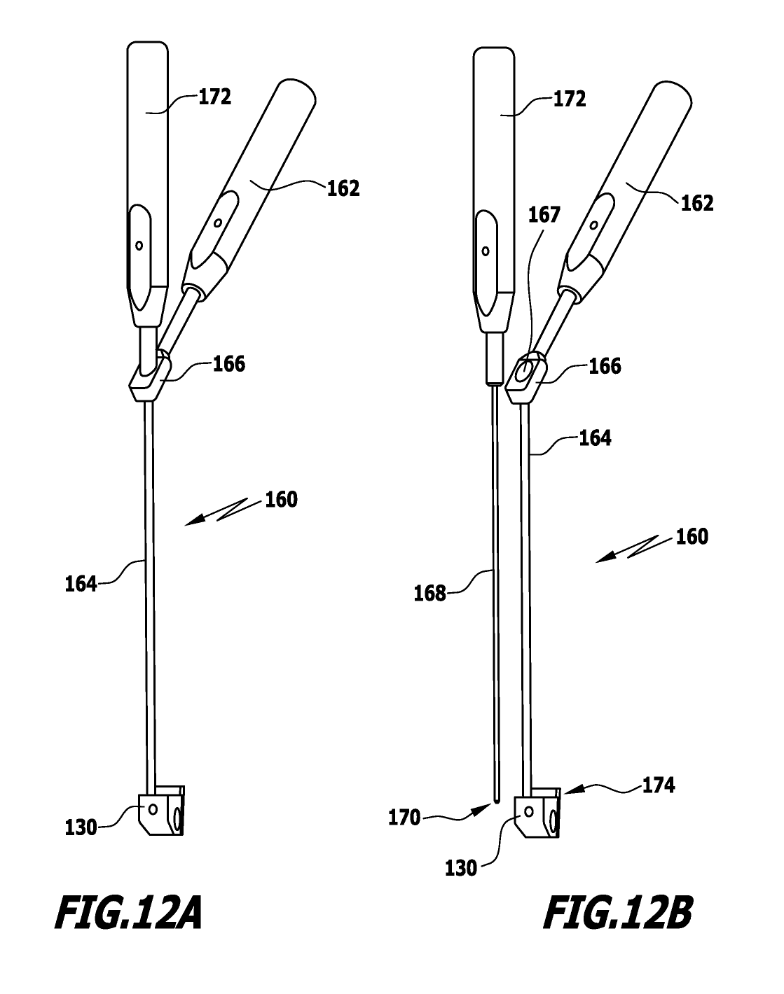

FIGS. 12A and 12B holding devices for an implant in the assembled and separated state;

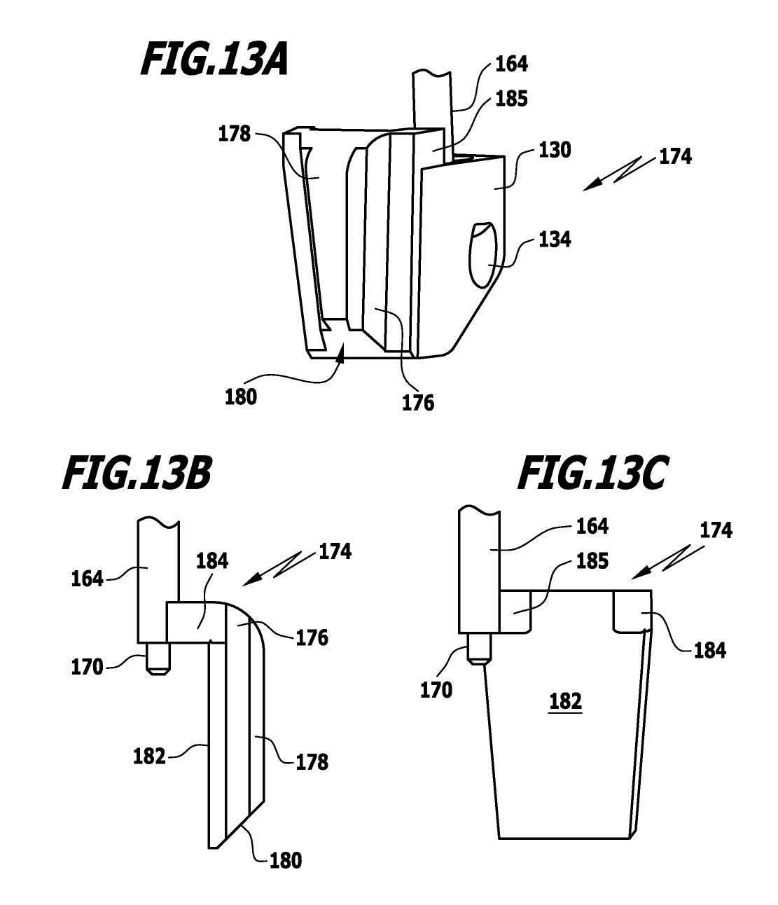

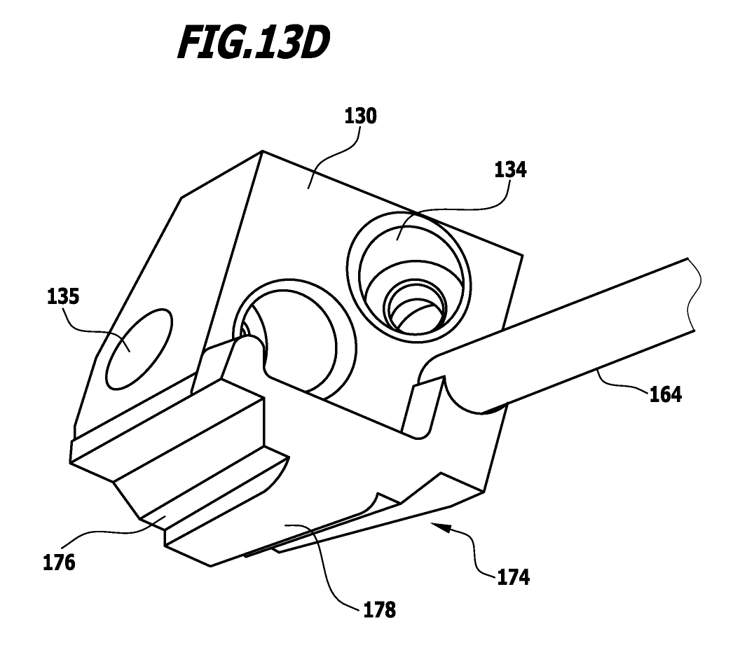

FIGS. 13A to 13D details of the holding device of FIG. 12 in various illustrations;

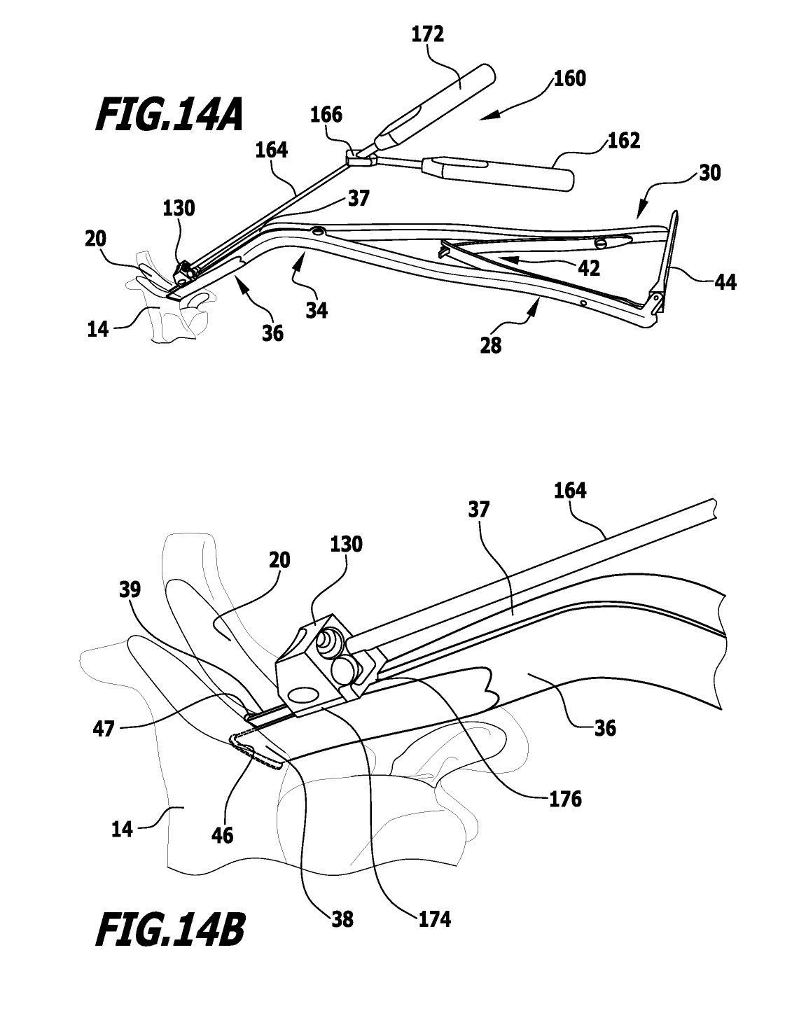

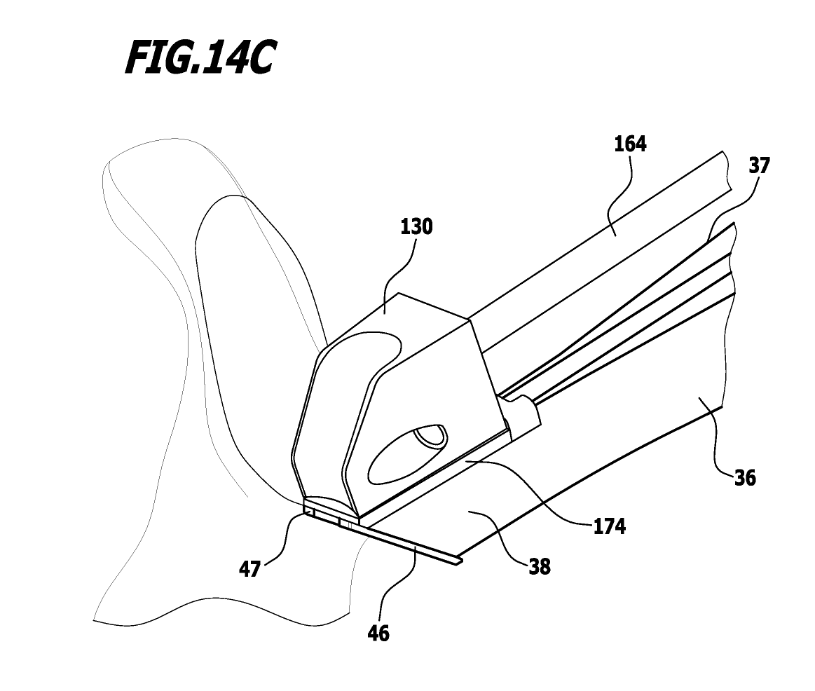

FIGS. 14A to 14C the holding device of FIG. 12 during insertion of an implant in an incision gap which is enlarged by a distraction instrument according to the invention of FIG. 2;

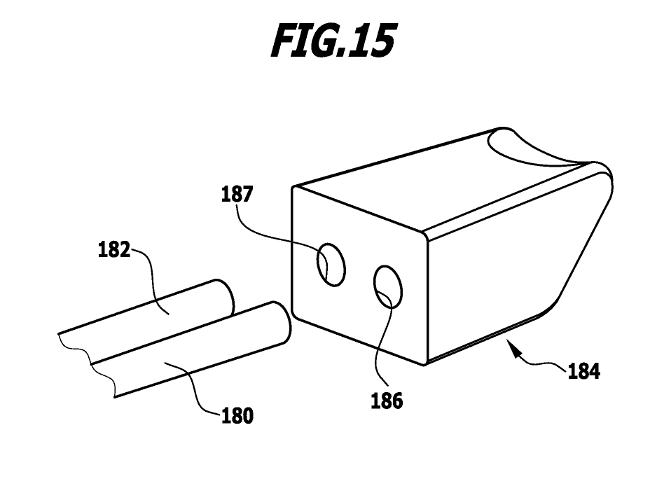

FIG. 15 an alternative holding device according to the invention for the implant;

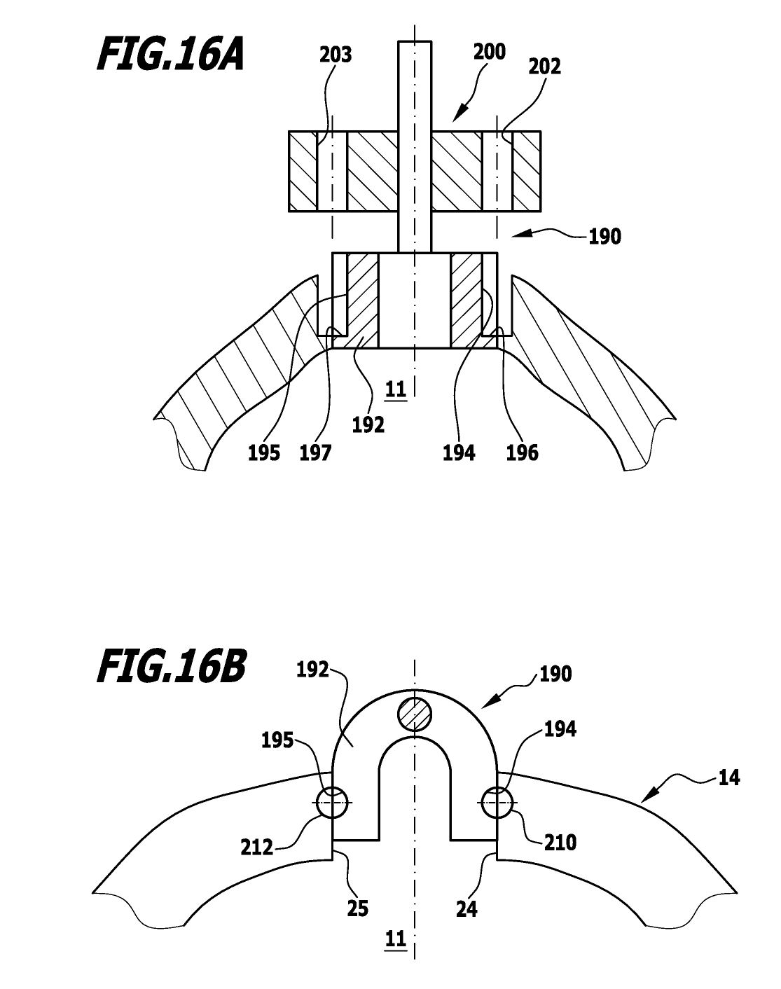

FIGS. 16A and 16B an alternative embodiment of the spacer according to the invention of FIG. 5; and

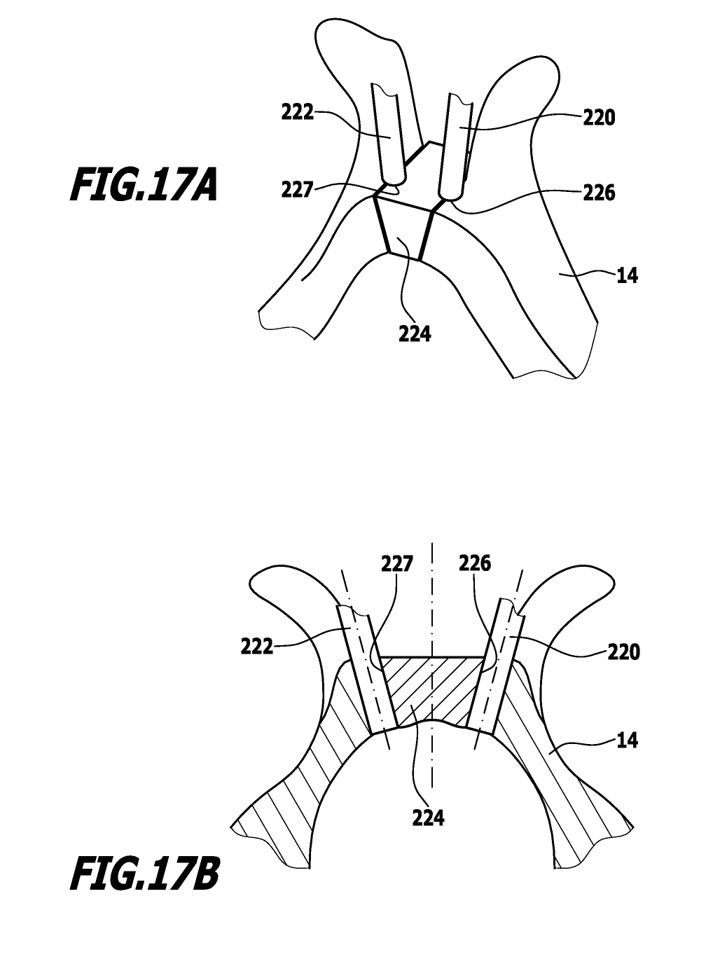

FIGS. 17A and 17B an additional, alternative distraction instrument according to the invention with inserted implant.

FIG. 1A shows in schematic representation the section C1 to C7 of a cervical spine 10 with a spinal canal 11 and a retractor 18 positioned over a vertebral arch 12 of a vertebra 14. The retractor 18 holds back the surrounding tissue (omitted in FIG. 1 for reasons of clarity) so that the area of the vertebra 14 remains dorsally accessible. The retractor forms part of instrumentation according to the invention.

Access to the vertebra 14 and its vertebral arch 12 is possible without detaching muscular tissue, thereby making its spinous process 15 accessible, whereas access to the lamina 16 requires detachment of muscular tissue.

In the state shown in FIG. 1A, it is possible to make a single incision gap 20 or 22 in the area of the lamina 16 or in the area of the spinous process 15 using an instrument (not shown) and this allows the vertebral arch 12 or its vertebral arch sections separated by the incision gap 20 or 22 to be elastically/plastically enlarged so that further areas of the vertebra need not be exposed.

The creation of the incision gap is not limited to any particular procedure. For example, the incision gap can be made with an ultrasonic osteotome, which enables particularly gentle splitting of the bone substance as far as the spinal canal 11. Here damage to the connective tissue of the spinal cord is avoided.

Alternatively, rapidly rotating drills may be used but the last phase of the splitting incision up to the spinal cord is preferably carried out with a bone punch.

A further alternative is offered by the so-called T-saw or Gigli saw, with which the incision gap is made, starting from the spinal canal 11.

FIG. 1B shows the vertebra 14 with the incision gap 20 completed in the spinous process. The alternative incision gap 22 in one of the laminas of the vertebral arch 12 is indicated in broken representation.

The incision gap 20 has two incision surfaces 24 and 25 arranged in parallel in the state shown in FIG. 1B. The gap width in this state depends on the technique used to make the incision gap and, for example, when an ultrasonic osteotome is used, is approximately 1 mm or less. An incision gap width of approximately 2 mm to 3 mm is typically obtained with rapidly rotating drills.

A distraction instrument 28 according to the invention is used to elastically/plastically enlarge the incision gap 20 and is inserted into the incision gap 20 approximately in the direction of the sagittal plane, as shown in FIG. 2C.

The distraction instrument 28 has a gripping part 30 with two gripping branches 32, 33 which are mounted on a lever mechanism 34 so as to be pivotable relative to one another and at the distal area of which branches 36, 37 are pivotable relative to one another and have tool elements 38, 39 held thereon, in particular integrally formed thereon.

The tool elements 38, 39 have a cross section which tapers conically in the pivoting direction towards their distal or free end or end area 40 and allows the tool elements 38, 39 to be introduced into an incision gap, which has previously been formed, without the need for greater forces.

The gripping elements are preferably tensioned relative to one another by means of a leaf spring arrangement 42 and held in a closed rest position, in which the two tool elements 38, 39 abut on one another, as shown in FIGS. 2A and 2B.

The gripping part 30 has, in addition, a locking mechanism 44, with which a step-like adjustment of the gripping branches 32, 33 may be realized, and deriving therefrom a step-like enlargement of the incision gap, thereby utilizing the viscoelastic properties of the bone substance of the vertebral arch.

Alternatively or in combination with the locking device 44, a gap width display may be realized and/or a force measuring device so that the gap width achieved on the tool element side can be read at the gripping part 30 or the force used during the enlargement can be used in a dosed manner.

Preferably, the locking device 44 automatically secures a widened position of the distraction instrument once reached.

In FIG. 2B, the tool elements 38, 39 and the distal ends of the branches 36, 37 holding them are shown in detail.

The tool elements 38, 39 preferably have at their end area 40 laterally projecting contact surfaces 46, 47 which abut on the spinal canal side of the vertebral arch sections during and following introduction of the tool elements 38, 39 into the incision gap 20, as shown in detail in FIG. 2C.

As a result, the distraction element 28 is guided on the wall of the vertebral arch sections on the spinal canal side and held in a position, in which the enlargement of the incision gap can be reliably brought about.

The results with respect to the enlargement of the vertebral canal diameter or the vertebral canal area can be explained using a simple model shown in FIG. 3.

The starting point is a C6 vertebra with the parameters indicated in FIG. 3 A=150.65 mm² and a diameter h of 11.5 mm. The calculations for a corresponding gap width x are based on the following assumptions:
The shape of the spinal canal in the vertebra can be approximated by a bent triangle 50 as shown in FIG. 3.
The pivot point 52 of the vertebral arch sections lies in the area of the so-called facet joints or small feet.
The sole elastic/plastic deformation of the sections of the vertebral arch 12 is assumed in the area of the lamina, wherein, for reasons of simplicity, its arch length has been taken to be constant and the bending lines 54, 55 have been simplified as curves.
The vertebral body (not shown) and the points, at which the lamina is connected to the vertebral body (small feet), are assumed to be rigid.

In the calculation, the width of the opening (gap width) x was increased in the range of 6 to 16 mm in 2 mm increments. The corresponding values for the increase in area ΔA and the increase in diameter Δh are listed in the following Table 1.

These values show that the value recommended in the literature (Wang, Xiang-Yang et al. in Spine, Vol. 31, No. 34, 2006, pages 2863 to 2870) for the increase in diameter can be achieved with the elastic/plastic deformation of the bone substance.

TABLE 1

| Width of gap | Increase in area | Increase in diameter |
|---|---|---|
| x | ΔA | Δh |
| 6 mm | 50.67 mm² | 3.79 mm |
| 8 mm | 63.30 mm² | 4.22 mm |
| 10 mm | 75.36 mm² | 4.53 mm |
| 12 mm | 86.72 mm² | 4.72 mm |
| 14 mm | 107.66 mm² | 4.99 mm |
| 16 mm | 126.84 mm² | 5.16 mm |

An alternative embodiment of a distraction instrument 60 according to the invention is shown in FIG. 4A with a gripping part 62, the gripping parts 64, 65 of which are connected to one another via a pivot bearing 66. Branches 68, 69 are integrally formed distally to the pivot bearing 66 and tool elements 70, 71 are held at their ends on the distal side. The free ends of the tool elements 70, 71 can, on the other hand, be designed as described for the preceding embodiment, can, in particular, be provided with contact elements which project laterally from the tool elements 70, 71.

In FIG. 4B, the tool elements 70, 71 are shown again in detail as well as contact surfaces integrally formed on them to the side in the form of flanges 72, 73. In contrast to the embodiment of the distraction instrument 28, the cross section of the tool elements in the distraction instrument 60 is not essentially rectangular but rather of a wedge-shaped design so that the width of the closed tool elements at their front side, i.e. the side first introduced into the incision gap, is smaller than at the end located opposite.

In addition, the distraction instrument 60 according to the invention has on its tool elements 70, 71 stop elements 74, 75 which project forwards and are integrally formed directly on the contact surfaces 72, 73.

Furthermore, the distraction instrument 60 according to the invention has on the upper side of the branches 68, 69 guide elements 76, 77 which can, for example, be manufactured from a plastic material.

The insertion of the distraction instrument according to the invention is shown on the basis of the distraction instrument 60 in FIG. 4C. FIG. 4C illustrates the introduction of the distraction instrument 60 in a sagittal direction, wherein the tool elements 70, 71 engage with their contact surfaces 72, 73 under the vertebral arches, i.e. abut on the vertebral arch of the vertebra 14 on the spinal canal side. This is illustrated again in detail in FIG. 4D.

Once the incision gap 20 is sufficiently enlarged, it can be stabilized in this state in accordance with a variation of the invention by a spacer 80 which is part of the instrumentation according to the invention, as shown in FIGS. 5A and 5B.

The spacer 80 preferably comprises a spacer element 82 bent in the shape of a U, which keeps the incision surfaces 24, 25 of the incision gap 20 at a prescribed spacing. In this state, the spinal canal is freely accessible in the area of the vertebra 14. The two legs 84, 85 of the U-shaped spacer element 82 are preferably configured so as to extend over a large area so that the contact between the incision surfaces 24, 25 of the incision gap 20 and the spacer element is over as large an area as possible and as small a surface pressure as possible results with the forces required for the elastic/plastic enlargement of the vertebral canal. The preferred measure of arranging the outside surfaces of the spacer element 82 in the shape of a wedge at an incline to each other in accordance with the inclination of the incision surfaces 24, 25 of the enlarged incision gap 20 also serves this purpose.

In order to hold the spacer element 82 as securely as possible in its position between the incision surfaces in the incision gap, the surfaces facing the incision surfaces 24, 25 have a plurality of parallel ribs 86, which counteract any unintentional sliding of the spacer element out of the incision gap in the dorsal direction. Owing to the parallelism of the ribs 86, once the implant has been inserted into the incision gap 20, the spacer element 82 can be removed from the incision gap 20 in the sagittal direction without applying any great force.

The spacer element can be additionally secured in its position, in which it is inserted in the incision gap, by a stem-like holder 88 which is preferably integrally formed on the arched part of the spacer element 82. When a retractor 18, as shown, for example, in FIG. 5A, is used, the holder 48 may, if required, be fixed to it.

FIG. 6A shows an alternative retractor in the form of a retractor sleeve 90 which is simply inserted into the tissue incision above the vertebra 14 to be operated and then the tissue around the operating site is held back with its cylindrical body 92.

In FIG. 6B, the retractor 90 is shown in its typical position of use (but without the surrounding tissue).

The retractor 90 with its cylindrical body or the sleeve 92 is, in some cases, completely adequate for keeping the operating area free as described. It has, in addition, the advantage that it does not extend into the surrounding area of the operating site and leaves this essentially freely accessible.

FIG. 6B shows the spacer 80 with the spacer element 82 positioned in the incision gap 20 with use of the retractor 90. The incision surfaces 24, 25 are kept at a predetermined distance while the U-shaped spacer element ensures free view and access to the spinal canal.

FIG. 6B shows in the plan view the access to the spinal canal 11 created by the elastically enlarged incision gap 20 which also results, of course, in the same way when using a retractor of a different type, such as, for example, the retractor 18.

FIGS. 7A and 7B show schematically in a side view and a plan view, respectively, the possibility of access to the spinal canal 11 with a so-called shaver 100 by way of example. Given the same gap width, in order to create a greater operating range for use of surgical instruments, for example a shaver or a punch, and to achieve further advantages which will be discussed hereinbelow, grooves 102, 103 are preferably formed in the incision surfaces 24, 25 of the incision gap 20 and it is further preferred for these to extend over the entire height of the incision surfaces 24, 25.

With the shaver 100, as shown in FIGS. 7A and 7B, it is, for example, possible to machine the lamina on the spinal canal side of the vertebral arch in order to create space for further decompression of the spinal cord.

This enables removal of bone substance on the spinal canal side of the lamina in order to increase its elasticity and allow further enlargement. In difficult cases, creation of a groove on sides of the spinal canal may also be considered so that, similarly to the conventional double-door technique, the vertebral arch sections can be bent about a kind of hinge. In contrast to the conventional technique, this does, however, not require any removal of muscle from the spine so that it is still a gentler surgical intervention.

Once the enlarged incision gap 20 is ready for insertion of an implant, an implant body 110 or 112, as shown, by way of example, in FIGS. 8A and 8B, is preferably inserted into the incision gap 20.

The two implant bodies 110, 112 are both of solid construction and are preferably produced from a plastic material suitable for implants, in particular PEEK. The two implant bodies 110, 112 have contacting surfaces 114, 115 inclined in relation to each other, which, in the inserted state of the implant, are in contact over as large an area as possible with the incision surfaces of the incision gap. While the implant bodies 110, 112 are of wedge-shaped configuration in a front view, it is quite possible for them to be of a substantially rectangular configuration in the side view.

The plastic material suitable for an implant (e.g. PEEK) is preferably coated, for example, by the Plasmapore technique or with a hydroxyapatite coating to promote growth of the bone substance on the implant.

At their upper end in FIGS. 8A and 8B, which, in the inserted state of the implant, lies dorsally, the implant bodies 110, 112 have laterally protruding projections 116, 117 which may assume several functions:

Firstly, the projections 116, 117 have the effect that the implant can only be inserted into the incision gap up to the point, at which the projections 116, 117 strike the bone substance, and a displacement of the implant in the direction of the spinal canal is also prevented in the postoperative phase.

Furthermore, the projections 116, 117 in positively locking engagement with an instrument or with corresponding recesses in the bone substance may act as a guide for the implant body during insertion of the implant into the incision gap and thereby assist precise placement.

Finally, the projections increase the size of the dorsal area of the implant body and therefore facilitate accommodation of holding elements such as, for example, screws, spikes, etc., which serve to fix the implant in the enlarged incision gap.

A bar 118 undercut on both sides is integrally formed as a gripping element on the implant bodies 110, 112 so as protrude dorsally therefrom. The bar 118 serves for easier handling during insertion and correct positioning of the implant.

FIG. 9 shows the procedure of inserting the implant body 112 into the incision gap 20, which is held by a spacer 80 in the enlarged state. In order to create sufficient space for insertion of the implant, the spacer 80 may be moved to some extent out of the incision gap in the sagittal direction so that only quite small surface areas of the legs of the spacer element 82 remain in contact with the incision surfaces 24, 25 of the incision gap 20. The implant is then inserted into the incision gap 20, as shown in FIG. 9, with forceps 120 at a slight incline to the incision gap axis.

After complete removal of the spacer 80, the implant body 112 is brought into its final position, as shown in FIG. 10. In this example, the height of the implant body 112 is selected such that, in the inserted state, it does not quite reach the spinal canal with its ventrally located end so that an additional volume remains there for the decompression of the spinal cord.

The illustrations A and B of FIG. 11 show a sectional view through an implant body 130 with two through-openings 134, 135 extending at an acute angle to the contacting surfaces of the implant body 132. The two through-openings 134, 135 are in staggered arrangement so that only the through-opening 134 located at the front in the drawing is fully visible in the sectional representations A and B of FIG. 11. Arranged in both through-openings 134, 135 are threaded bolts 136, 137, of which the threaded bolt 137 is already fully screwed into the through-opening 135 and with its pointed end penetrates beyond the contacting surface of the implant 130 into the bone substance of a vertebral arch (not shown here).

In illustration A of FIG. 11, the threaded bolt 136 is still shown in its initial position, in which it is held with positive locking by a short thread section 138 forming part of the through-opening 134.

After insertion of the implant body 132 into an incision gap of a vertebral arch, the two threaded bolts 136, 137 are screwed in and the implant 130 thereby fixed to the surrounding bone substance. The thread section 138 together with the remaining parts of the through-openings 134, 135 guides the threaded bolts so that they can penetrate the surrounding bone substance with a predefined orientation.

The through-openings 134, 135 have a larger diameter at their dorsally located openings at the surface of the implant 130 so that in the screwed-in state the threaded bolts 136, 137 can be fully accommodated with their bolt head 140 within the body of the implant 130. This area 139 of extended diameter of the through-opening 134, 135 is followed by the previously mentioned section with an internal thread 138.

The threaded bolts 136, 137 are preferably so configured that a so-called free-running section 142 is provided at their area adjacent to the bolt head 140 and this is long enough to pass through the internal thread 138 of the through-opening 134, 135 so that when the threaded bolts 136, 137 (FIG. 11B) are fully screwed in the thread of the threaded bolts becomes disengaged from the internal thread 138 and, as a result, by tightening the threaded bolts 136, 137, the implant body 132 can be made to bear tightly with its contacting surfaces against the incision surfaces of the bone substance.

The length of the threaded bolts 136, 137 is of such dimensions that even after they have been screwed fully into the implant body 132 they do not protrude far enough out of the implant body to be able to penetrate the spinal canal. In the fully screwed-in state, the point of the threaded bolts 136, 137 is, therefore, preferably positioned so as to remain behind the ventral front edge 144 of the implant 130.

FIG. 11C shows the implant 130 of FIG. 11A in a perspective illustration with its ventral or rather distal end area 144 (front edge) which is provided with an indentation so that additional volume is created adjacent to the spinal canal.

In the alternative of FIG. 11D, the implant 150 has an essentially similar structure to the implant 130 with the difference that a nose 154 is integrally formed at the ventral end area 152 and this can engage between the branches of distraction forceps during introduction of the implant into the incision gap of the vertebral arch so that the implant 150 can be introduced into the incision gap of the vertebral arch in a guided, in particular centered, manner.

In order to be able to handle the implants according to the invention better, they are preferably attached to a holding and insertion instrument which is part of the instrumentation according to the invention. Preferred implants have corresponding features for the connection to the holding and insertion instrument, for example a bore in the dorsally located end surface 145, as illustrated in FIGS. 11A and 11B with the example of bore 146 with internal thread. A holding and insertion instrument can be fixed in it parallel to the longitudinal direction of the implant which will be explained in greater detail in conjunction with FIGS. 12 to 14.

At its ventral end 144, the implant body 130 has an indentation 148 which extends around the ventrally protruding edge and allows a further increase in the size of the space available on the spinal canal side and, therefore, further decompression of the spinal cord. With this measure, the implant body 130 enables an additional gain in space for the spinal canal which could otherwise only be achieved by a substantially greater spreading of the vertebral arch.

FIGS. 12A and B show a holding and insertion instrument 160 with a handle 162 and a shaft 164 projecting at an angle from its distal end. The shaft 164 is designed as a tube-like part and is connected with its proximal end to the handle 162 via a connection piece 166 which has, flush with the shaft 164, a through-bore 167, via which the hollow space in the shaft 164 is accessible, on the side facing away from the shaft.

The holding and insertion instrument 160 has, in addition, a threaded shaft 168 which can be inserted into the hollow cylindrical shaft 164 via the through-opening 167 and which reaches as far as the distal end of the shaft 164.

The threaded shaft 168 has a threaded section at its distal end 170 while a handle 172 is fixed coaxially to its proximal end. Finally, a slide 174 is mounted on the distal end of the shaft 164 and this forms a receptacle for an implant 130.

As soon as the implant 130 is positioned on the slide 174, the threaded shaft 168 can be inserted into the through-opening 167 and the shaft 164, respectively, and its threaded section screwed into the bore 146 of the implant 136 at the distal end 170.

In this state, the handle 172 is supported on the connecting piece 166 and thus fixes the implant 130 in position on the slide 174.

Details of the slide 174 are illustrated in FIGS. 13A to 13D. In FIGS. 13A and 13D, the implant 130 is mounted on the slide 174, in FIGS. 13B and 13C the slide 174 is illustrated as such. A centering projection 176 is provided on the rear side of the slide 174 illustrated to the front in FIG. 13A and this projection is provided for guiding the slide between the branches of a distraction instrument according to the invention. The projection 176 preferably has centrally and in longitudinal direction a projecting shoulder 178 which serves to center the slide preliminarily between the branches of the distraction instrument.

At its distal end, the slide 174 has an inclined insertion surface 180 which serves for the introduction of the slide 174 with the implant 130 into the enlarged incision gap 120 or makes this easier. The inclined insertion surface 180 is inclined at an angle of approximately 45° relative to a contact surface 182 for the implant 130. At its proximal end, the slide 174 has two stoppers 184, 185 which project over the contact surface 182, abut on the dorsal end surface 145 of the implant body 130 and thus define its position on the contact surface 182.

The stopper 185 is then securely connected to the shaft 164 of the holding and insertion instrument 160. In FIGS. 13B and 13C, the threaded section of the distal end 170 of the threaded shaft 168 of the insertion and holding instrument 160 is to be seen protruding at the lower end of the shaft 164.

FIG. 13D shows the implant 130 mounted on the slide 174 approximately as viewed by the surgeon.

FIGS. 14A to C show the use of the holding and insertion instrument 160 according to the invention with mounted implant 130 in conjunction with a distraction instrument 28 placed in the incision gap 20 of a vertebra 14. The implant 130 on the slide 174 slides along the branches 36, 37 and is thereby centered between the branches 36, 37 by the projection 176.

It is easy to handle the implant 130 with this holding and insertion instrument 160. This holding and insertion instrument 160 will preferably be used together with distraction forceps or a distraction instrument as shown in FIG. 4A, wherein the implant 130 itself is then guided between the guide elements provided in this case on the upper side of the branches 68, 69.

In the situation illustrated in FIG. 14C, in which one part of the vertebral arch, which would have been at the front, is omitted for the purpose of illustrating the end position of the inserted implant 130, the implant 130 or rather the slide 174 is shown positioned on the contact surfaces 46, 47 of the tool elements 38, 39. Only in this state, i.e. when the implant is arranged in its end position in the incision gap, will the holding and insertion instrument 160 be released in that the threaded shaft 168 is turned out of the bore 146 of the implant 130 with its end 170 so that, subsequently, the slide 174 with the holding and insertion instrument 160 can be withdrawn from the field of operation.

Thereafter, the distraction instrument 28 can also be removed from the incision gap 20, wherein this is again removed in a sagittal direction.

If the implant 130 is still not arranged in the correct position in the incision gap 20 when the slide 174 reaches the contact surfaces 146, this will be turned about the contact point into the correct position.

FIG. 15 shows schematically an additional possibility of a holding and insertion instrument with two cylindrical branches 180, 182 together with an implant body 184 which has two bores 186, 187 on the dorsal side, in which the branches 180, 182 are accommodated.

FIGS. 16A and 16B show an alternative spacer 190 which has a U-shaped spacer element 192 similar to the spacer 80.

Semicylindrical recesses 194, 195 are provided in the externally located surfaces of the U-shaped spacer element 192 and these extend over a large portion of the height of the spacer element 192 but are closed towards the bottom (towards the spinal canal) via a stop 196, 197. These semicylindrical openings 194, 195 offer together with a boring gauge 200, which has through-bores 202, 203 aligned relative to the semicylindrical recesses 194, 195, an exact guidance for a drilling tool (not illustrated), with which semicylindrical recesses can be introduced into the bone substance in the incision surfaces of the incision gap.

The working ends 220, 222 or tool elements of a distraction instrument may be inserted into these bores 210, 212, this instrument keeping the incision gap first of all in the enlarged position. Subsequently, an implant 224 of a corresponding design can be inserted and, preferably, this likewise has semicircular grooves 226, 227 in the contact surfaces. The working ends 220, 222 or tool elements of a distraction instrument will be guided along via these semicylindrical grooves 226, 227 until it is inserted in the incision gap.

Such a situation is shown in FIGS. 17A and 17B.

Subsequently, the distraction instrument with its cylindrical working ends 220, 22 can be withdrawn and the remaining bores 210, 214 then serve for the insertion of screws or dowels (not shown) for fixing the implant in place in the incision gap.

1. Surgical distraction instrument for use in laminoplasty, wherein the vertebral arch of a vertebra is cut through, thereby forming an incision gap, and the incision gap is enlarged with the distraction instrument for the purpose of inserting a, in particular, wedge-shaped implant, the distraction instrument comprising: a proximal gripping part and two distal branches movable relative to one another with tool elements held thereon, a lever mechanism arranged between the gripping part and the branches, wherein the tool elements are held with the lever mechanism so as to be movable relative to one another from a closed rest position into an open spread position.

2. Instrument as defined in claim 1, wherein the tool elements have a contact surface at their distal end for the positioning of the instrument in the gap.

3. Instrument as defined in claim 1, wherein the tool elements have a stop element at their distal end for the positioning of an implant in the incision gap.

4. Instrument as defined in claim 1, wherein the tool elements have an end area insertable into the incision gap, the cross section of said end area being of a wedge-shaped design.

5. Instrument as defined in claim 1, wherein the tool elements are designed so as to taper conically towards their distal end.

6. Instrument as defined in claim 1, wherein the tool elements each have a bending strength corresponding to a distraction force of approximately 300 N or more without any plastic deformation occurring at the tool elements.

7. Instrument as defined in claim 1, wherein the longitudinal axes of the branches holding the tool elements are arranged at an obtuse angle with respect to the longitudinal axis or plane of pivoting of the gripping part.

8. Instrument as defined in claim 6, wherein the angle is approximately 110° to approximately 160°.

9. Instrument as defined in claim 1, wherein the branches comprise guide elements.

10. Instrument as defined in claim 1 to 9, wherein the instrument has a locking device for fixing the tool elements in one or more predetermined relative positions.

11. Instrument as defined in claim 1, further comprising a device for determining the distance between the end areas of the tool elements.

12. Instrument as defined in claim 1, further comprising a force measuring device.

13. Instrument as defined in claim 12, wherein the force measuring device is designed with a display for a predetermined maximum force.

14. Surgical instrumentation for expanding a vertebral canal of a vertebra, comprising:
a distraction instrument, the distraction instrument comprising a proximal gripping part and two distal branches movable relative to one another with tool elements held thereon, a lever mechanism arranged between the gripping part and the branches, wherein the tool elements are held with the lever mechanism so as to be movable relative to one another from a closed rest position into an open spread position; and
a holding instrument for an implant to be inserted.

15. Instrumentation as defined in claim 14, wherein the holding instrument has a proximally arranged gripping part, a shaft adjoining thereto and a holding device arranged at the distal end of the shaft for releasably fixing an implant in position.

16. Instrumentation as defined in claim 15, wherein the holding device has a slide preferably designed complementarily to the guide elements of the distraction instrument.

17. Instrumentation as defined in claim 14, further comprising a spacer preferably comprising a spacer element of a U-shaped design.

18. Instrumentation as defined in claim 17, wherein the spacer is designed as a drilling gauge.

19. Instrumentation as defined in claim 17, wherein the spacer has a shaft for the extracorporeal fixing in position.

20. Instrumentation as defined in claim 14, further comprising a retractor designed such that the spacer is adapted to be secured to the retractor with its shaft.
